(19) United States
(12) Patent Application Publication
(10) Pub. No.: US 2023/0040339 A1
(43) Pub. Date: Feb. 9, 2023

(54) THERAPEUTIC AMOEBA AND USES THEREOF

(71) Applicant: AmebaGone, LLC, Madison, WI (US)

(72) Inventors: Marcin Filutowicz, Madison, WI (US); Chad Hall, Madison, WI (US); Amy Jancewicz, Madison, WI (US)

(21) Appl. No.: 17/788,209

(22) PCT Filed: Dec. 22, 2020
(86) PCT No.: PCT/US2020/066606

§ 371 Date: Jun. 22, 2022

Related U.S. Application Data
(60) Provisional application No. 62/952,662, filed on Dec. 23, 2019.

(51) Int. Cl. A01N 63/10; A61K 45/06

(52) U.S. Cl. CPC A01N 63/10; A61K 45/06

(57) ABSTRACT

The present disclosure relates to amoebae (slime molds) and uses thereof. In particular, the present disclosure relates to the use of amoebae or their environmentally stable spores to control agricultural infections and other uses.

This application claims priority to U.S. provisional patent application Ser. No. 62/952,662, filed Dec. 23, 2019, which is incorporated herein by reference in its entirety.

### FIELD OF THE DISCLOSURE

The present disclosure relates to amoebae (slime molds) and uses thereof. In particular, the present disclosure relates to the use of amoebae or their environmentally stable spores to control agricultural infections and other uses.

### BACKGROUND OF THE DISCLOSURE

Farmers across the globe today must produce more food than ever to meet the growing demands of the largest human population that has ever inhabited the Earth. In order to meet this challenge, growers use millions of pounds of chemicals annually to protect crops against pests and diseases that devastate crop yield and quality. Even with chemical controls, estimates of annual crop losses still hover around 40% of the potential yield.

FireBlight (FB) causes devastating impacts on growers of pone fruits (e.g., apples, pears, and quince). Caused by the bacterial pathogen Erwinia amylovora (Ea), FB usually infects trees via insects traveling between blossoms during a 2-week period in the springtime. Once Ea has established in the flower, it can travel into vascular tissues, down the branches, and into the trunk. Leaves develop a burned appearance, hence the disease's name. Fruitlets and branches develop brown water-soaked lesions and become necrotic. Once present, the only means to control the disease is to stop its spread, meaning growers must prune and destroy infected tissue, which takes years to decades to establish.

For organic growers, who are restricted from using certain chemicals in the protection of their crops, the problem is dire. In 2014, the U.S. National Organics Standards Board recommended phasing out use of streptomycin and oxytetracycline that have been relied on in organic apple and pear production for control of FB, causing an immediate need for organic alternatives. FB serves as just one example of the many bacterial diseases that threaten agricultural crops. Ea serves as just one example of the 10 most common phytopathogens that are capable of destroying the crops (Mansfield, J., et al. (2012). “Top 10 plant pathogenic bacteria in molecular plant pathology.” Molecular plant pathology 13(6): 614-629).

What is needed are new treatments for microbial infections in plants.

### SUMMARY OF THE DISCLOSURE

The present disclosure relates to amoebae (slime molds) and uses thereof. In particular, the present disclosure relates to the use of amoebae or their environmentally stable spores to control agricultural infections and other uses.

For example, in some embodiments, provided herein is a method of treating a microbial infection in a plant, comprising: contacting the plant with a composition comprising one or more species of amoebae. In some embodiments, the microorganism is a bacteria (e.g., including but not limited to, Erwinia amylovora (Ea), Clavibacter michiganensis subsp (Cm). Michiganensis (Cmm), Xanthomonas campestri (Xc)s, or Pseudomonas syringae Ps)). In some embodiments, the target bacteria are present as a biofilm. In some embodiments, the composition comprises two or more species of amoebae. In some embodiments, the amoebae are a Dictyostelium sp (e.g., including but not limited to, Illinois 15b, RI-1, WS-606, Cohen 22, NC-4, WS-28-1, WS-331a, Wychwood 4, WS-15, WS-20, WS-28, WS-142, WS-647, DC-60, or Cohen 36). In some embodiments, the two or more amoebae are WS606+Illinois 15b or DC60+Illinois 15b. In some embodiments, the composition further comprises a non-amoebae anti-microbial agent (e.g., including but not limited to, one or more of a pesticide, an insecticide, a fertilizer, a herbicide, or a fungicide). In some embodiments, the treating reduces one or more signs or symptoms of infection selected from, for example, blossom blight, cankers or necrosis.

Additional embodiments provide a composition, kit, or system, comprising: a) one or more species of amoebae; and b) a carrier. In some embodiments, the amoebae are present as rehydrated lyophilized spores, or are in another life stage. In some embodiments, the composition is formulated as a dip, seed dressing, stem injection, spray, or mist. In some embodiments, amoebae are present in unit dosage form. In some embodiments, amoebae are at a concentration of 1×10⁶ spores per ml or higher.

Additional embodiments are described herein.

### DESCRIPTION OF THE FIGURES

FIG. 1 shows dictyostelid Developmental Cycle. A) Cells from the genus Dictyostelium exist as free-living amoebae but are able to form multicellular aggregates that then develop into structures that support the formation of environmentally-resistant spores within a fruiting body. The spores are able to germinate to resume feeding as free-living amoebae upon the introduction of more favorable conditions. B) Macro- and Microscopic images of stages of Dictyostelid development.

FIG. 2 shows quantitative measure of Dicty feeding on detached leaves from Jonathan apple trees. A) 31 Dicty strains were tested for their ability to destroy Ea on detached leaves from Jonathan apple trees. The table shows all 31 Dicty strains. B) Dicty can form sporangia (reproductive structures) when inoculated onto leaves of apple trees indicating that leaves can support Dicty growth and Dicty is not harmful to leaves.

FIG. 3 shows results of a qualitative pear core assay for Dicty strains produced on Ea.

FIG. 4 shows quantitative measure of in planta Dicty efficacy against FB in whole Jonathan apple trees infected via a blossom route of infection.

FIG. 5 shows that Dicty reduces blossom Blight in Field trials.

FIG. 6 shows titer reduction of Ea in seedlings treated with individual and combinations of Dicty strains.

FIG. 7 shows that large-scale production yielded trillions of spores for field trials. A) Cake pan used to grow large-scale DC60 spores. B) harvested Illinois 15b spores. C) Dicty were grown on E. coli B/R on custom low ionic strength agar in the plastic lids of 8″×13″ cookie sheets.

FIG. 8 shows that Dicty feed on Xc and Ps.

FIG. 9 shows number of diseased blossoms after different treatments.

FIG. 10 show percent of blighted floral clusters after different treatments.

### DEFINITIONS

To facilitate an understanding of the present disclosure, a number of terms and phrases are defined below.

As used herein, the term “pathogen” refers to a biological agent that causes a disease state (e.g., infection, canker, etc.) in a host. “Pathogens” include, but are not limited to, bacteria, fungi, archaea, and the like.

As used herein, the term “microorganism” refers to any species or type of microorganism, including but not limited to, bacteria, archea, fungi, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are gram negative or gram positive. “Gram negative” and “gram positive” refer to staining patterns with the Gram-staining process that is well known in the art. (See e.g., Finegold and Martin, Diagnostic Microbiology, 6th Ed., C V Mosby St. Louis, pp. 13-15 [1982]). “Gram positive bacteria” are bacteria that retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red. In some embodiments, the bacteria are those capable of causing disease (pathogens) and those that cause production of a toxic product, tissue degradation or spoilage.

As used herein, the term “fungi” is used in reference to eukaryotic organisms such as the molds and yeasts, including dimorphic fungi.

As used herein the term “biofilm” refers to an aggregation of microorganisms (e.g., bacteria) surrounded by an extracellular polymeric substance (EPS) (Flemming, H. C., et al. (2007). “The EPS matrix: the “house of biofilm cells”.” J Bacteriol 189(22): 7945-7947.) or slime adherent on a surface in vivo or ex vivo, wherein the microorganisms adopt altered metabolic states rendering them tolerant to antibiotics and disinfectants.

The term “diagnosed,” as used herein, refers to the recognition of a disease by its signs and symptoms (e.g., appearance of a plant), or genetic analysis, pathological analysis, histological analysis, diagnostic assay (e.g., for microorganism infection) and the like.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., a plant) and to processes or reaction that occur within a natural environment.

As used herein, the term “host cell” refers to any eukaryotic or prokaryotic cell (e.g., plant cells), whether located in vitro or in vivo.

As used herein, the term “genome” refers to the genetic material (e.g., chromosomes or plasmids) of an organism or a host cell.

As used herein, the term “effective amount” refers to the amount of a therapeutic agent (e.g., an amoeba) sufficient to effect beneficial or desired results. An effective amount can be administered in one or more administrations, applications or dosages and is not intended to be limited to a particular formulation or administration route.

The term “sample” as used herein is used in its broadest sense. A sample may comprise a cell, tissue, or fluids, nucleic acids or polypeptides isolated from a cell (e.g., a microorganism), and the like.

As used herein, the terms “purified” or “to purify” refer, to the removal of undesired components from a sample. As used herein, the term “substantially purified” refers to molecules that are at least 60% free, preferably 75% free, and most preferably 90%, or more, free from other components with which they are usually associated.

As used herein, the term “modulate” refers to the activity of a compound (e.g., an amoebae) to affect (e.g., to kill or prevent the growth of) a microorganism.

### DETAILED DESCRIPTION OF THE DISCLOSURE

The present disclosure relates to amoebae (slime molds) and uses thereof. In particular, the present disclosure relates to the use of amoebae or their environmentally stable spores to control agricultural infections and other uses.

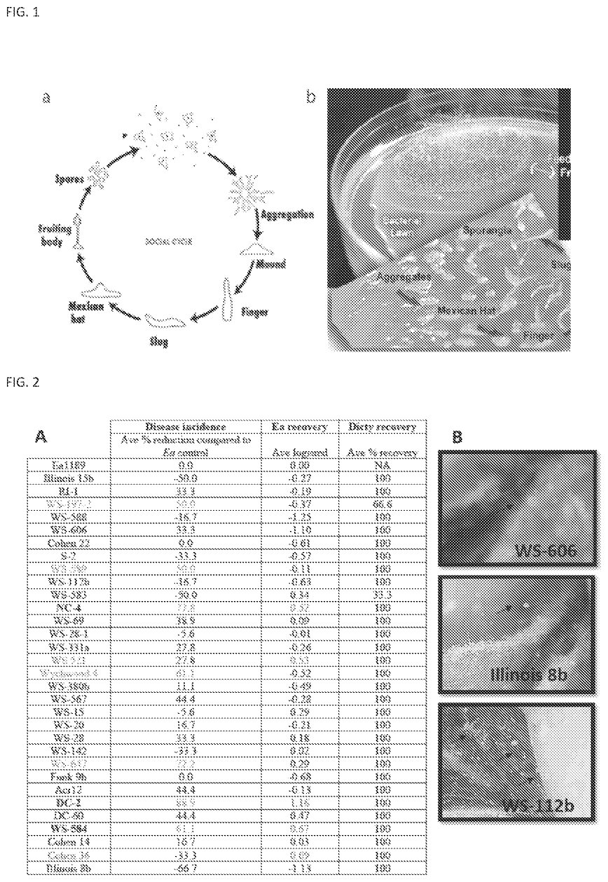

Experiments described herein identified strains of Dictyostelids (Dicty, FIG. 1)¹ as a biological treatment for bacterial infections of plants. Specific experiments identified strains and combinations of strains that treat the bacterial pathogen Ea that causes FB^{2,3} and Cmm that causes bacterial canker in tomatoes⁵.

Embodiments of the present disclosure provide for the use of amoebae (slime molds) in treatment and prevention of microbial infection, in particular, against some of the most tenacious pathogens. Over close to 1 billion years amoebae have evolved to safely kill a broad range of pathogenic bacteria. They eat pathogens while leaving no toxic debris. The utility of amoebic therapy derives from amoebae (or their spores) being an easily transported and applied antibacterial agent, effective against a broad range of pathogens including drug resistant bacteria.

The ability of slime molds to feed on bacteria and fungi is described (Raper K B. 1984. The Dictyostelids. Princeton University Press. Princeton N.J.; Old, K. M. et al., .1985 Fine structure of a new mycophagous amoeba and its feeding on Cochliobolus sativus; S. Chakraborty, et al., 1985, Canadian J. of Microb, 31:295-297; Soil Biology and Biochemistry Vol 17, 645-655; A Duczek, L J % A Wildermuth, GB 1991 J Australasian Plant Pathology Vol 20, 81-85). When a few spores are added to bacteria growing on a plate, in a matter of hours they split open (germinate) and from each spore emerges a single amoeba that immediately begins to feed on the surrounding bacteria. As they grow, they divide into two (e.g., approximately every three hours) so vast numbers of amoebae are soon present. The soil-born amoebae feed first as independent amoebae. Each individual amoeba surrounds a bacterium (or other microorganism) with its pseudopods, encases it in a food vacuole, and extracts the needed nutrients. Thus, amoebae can be viewed as professional phagocytes that are similar to macrophages and neutrophils (Chen G, et al. 2007. Science. 317:678-68). Mechanistically, both amoebae and the immune cells capture bacteria by phagocytosis within cytoplasmic vesicles. These vesicles fuse with lysosomes as a step in the killing of entrapped bacteria. Once amoebae clean an area of bacteria, they then come together and aggregate to form a unit similar to a multi-cellular organism. During the social cycle, thousands of amoebae aggregate in tune to a camp signal and the aggregated cells can form a slug (FIG. 1). Ultimately the slug develops into spore-laden fruiting bodies. The social amoebae belonging to the phylum Mycetozoa have been described as primitive eukaryotes that exhibit characteristics found among both protozoans and fungi (Bonner J T. (2009); Raper K B, Rahn A W. (1984) The Dictyostelids). This description can be summarized in an illustration of their asexual life cycle. Each species of amoeba has a vegetative phase where, as microscopic independent amoeboid cells feed upon bacteria, grow, and multiply. When the amoebae exhaust their bacterial food source, they enter a social phase in which individual cells stream together to form a multicellular, differentiated, assemblage (in a phylogenetic group 4 of Dictyostelids (Sheikh S., Thulin, M Cavender, J C. Escalante, R., Kawakami, S, et al. (2017). “A new Classification of the Dictyostelids.” Protist (in press) mobile slug. Since growth occurs at the single-cell stage, its size depends on how many amoebae have entered the aggregate, and slugs will vary in length from about 0.2 to 2 millimeters, a ten-fold range, and by the latest estimates the number of amoebae they contain ranges from about 10,000 to 2 million. The slug eventually comes to rest and develops into a macroscopic fruiting body consisting of a stalk with sorocarp. Within the sorocarp are environmentally and temporally stable spores, which are disseminated by the wind, animals, or the forces generated by the sorocarp falling. From each viable spore a single amoeba arises.

Unlike animals or plants, amoebae eat first; then grow by simply producing an increasing number of separate amoebae, and when food (bacteria/fungi) is gone they stream together to become multi-cellular. Once amoebae form their fruiting bodies they can no longer do anything that requires an intake of energy: they are static. The only part of them that is alive is the dormant spores.

In addition to their feeding behavior, amoebae possess many other virtues that are conducive to an amoebic antimicrobial treatment: Most prominent virtues of this group of organisms have been studied and extensively described for Dictyostelium discoideum. Although the below discussion in exemplified by D. discoideum, the present disclosure is not limited to a particular strain of Dictyostelid amoeba.

D. discoideum amoebae and spores themselves are not known to be pathogenic to animals and plants. D. discoideum consumes and digests a variety of pathogenic and non-pathogenic bacteria, whether live, dormant or dead. Moreover, bacteria that are resistant to conventional antibiotics are consumed by D. discoideum (See e.g., Smith M G, et al. 2007. Genes Dev. 21:601-614). D. discoideum not only kills free bacteria, but can consume bacteria living as a colony or biofilm (Raper K B. 1984. The Dictyostelids. Princeton University Press. Princeton N.J.; Sanders, D., et al. (2017). “Multiple Dictyostelid Species Destroy Biofilms of Klebsiella oxytoca and Other Gram Negative Species,” Protist 168(3): 311-325.). Thus, slime molds further find use in controlling microbial biofilms (Sanders, D., et al. (2017). “Multiple Dictyostelid Species Destroy Biofilms of Klebsiella oxytoca and Other Gram Negative Species.” Protist 168(3): 311-325). As a eukaryotic organism, D. discoideum amoeba is not susceptible to anti-prokaryotic antibiotics. Therefore, amoebae can be used in conjunction with most of the antibiotics used to treat bacterial infections.

In some embodiments, amoebic treatment utilizes overwhelming numbers of amoebae. Locally, these amoebae quickly ingest and digest their bacterial prey. The present disclosure is not limited to a particular mechanism. Indeed, an understanding of the mechanism is not necessary to practice the present disclosure. Nonetheless, it is contemplated that in the time frame of treatment, resistance to amoebae will be difficult for pathogens to acquire, and spread of resistance will be minimized. Certain bacteria are facultative intracellular pathogens and there are known strain of genetically engineered bacteria, like the benign soil bacterium Bacillus subtilis harboring the gene for lysteriolysin O, can survive within macrophage-like cell line (Bielecki J, et al. 1990. Nature, 345:175-176). However, in combination with more than one amoebae type or in combination with conventional antibiotics, resistance to amoebic therapy can be minimized or eliminated.

As described above, embodiments of the present disclosure provide compositions and methods for treating infection by microorganisms with amoebae. Examples of amoebae suitable for use in embodiments of the present disclosure include, but are not limited to, amoebae of the phylum Mycetozoa, which include but are not limited to: Dictyostelium: D. laterosorum, D. tenue, D. potamoides, D. minutum, D. gracile, D. lavandulum, D. vinaceo-fuscum, D. rhizopodium, D. coeruleo-stipes, D. lacteum, D. polycephalum, D. polycarpum, D. polycarpum, D. menorah, D. caveatum, D. gloeosporum, D. oculare, D. antarcticum, D. fasciculatum, D. delicatum, D. fasciculatum, D. aureo-stipes var. helveticum, D. granulophorum, D. medusoides, D. mexicanum, D. bifurcatum, D. stellatum, D. microsporum, D. parvisporum, D. exiguum TNS-C-199, D. mucoroides, D. sphaerocephalum, D. rosarium, D. clavatum, D. longosporum, D. macrocephalum, D. discoideum, D. discoideum AX4, D. intermedium, D. firmibasis, D. brunneum, D. giganteum, D. robustum, D. multi-stipes, Dermamoeba algensis, D. brefeldianum, D. mucoroides, D. capitatum, D. pseudobrefeldianum, D. aureocephalum, D. aureum, D. septentrionalis, D. septentrionalis, D. implicatum, D. medium, D. sphaerocephalum, D. rosarium, D. clavatum, D. longosporum, D. purpureum, D. macrocephalum, D. citrinum, D. dimigraformum, D. firmibasis, D. brunneum, D. giganteum, D. monochasioides, Thecamoeba similis and Polysphondylium: P. violaceum, P. filamentosum, P. luridum, P. pallidum, P. equisetoides, P. nandutensis YA, P. colligatum, P. tikaliensis, P. anisocaule, P. pseudocandidum, P. tenuissimum, P. pallidum, P. asymmetricum, P. filamentosum, P. tenuissimum, P. candidum, Acytostelium; A. ellipticum, A. anastomosans, A. longisorophorum, A. leptosomum, A. digitatum, A. serpentarium, A. subglobosum, A. irregularosporum, Acraside; A. granulate, A. rosea; Copromyxa: C. protea, C. arborescens, C. filamentosa, and C. corralloides; Guttulina (Pocheina) G. rosea; Guttulinopsis G. vulgaris, G. clavata, G. stipitata, G. nivea (See e.g., Schaap, et al. 2006 Molecular Phylogeny and Evolution of Morphology in the Social Amoebas, Science 27 Oct. 2006: 661-663; Raper K B. 1984. The Dictyostelids. Princeton University Press. Princeton N.J.; each of which is herein incorporated by reference in its entirety).

In some embodiments, the amoebae are Dictyostelium sp (e.g., including but not limited to, Illinois 15b, RI-1, WS-606, Cohen 22, NC-4, WS-28-1, WS-331a, Wychwood 4, WS-15, WS-20, WS-28, WS-142, WS-647, DC-60, or Cohen 36). In some embodiments, the amoebae are WS606+Illinois 15b or DC60+Illinois 15b.

The amoebae described herein have evolved to consume a myriad of species of bacteria that live in soil communities. Like macrophages and neutrophils, single celled amoebae chase, engulf and digest their microbial prey (Chen G, Zhuchenko O, Kuspa A. (2007) Science 317(5838): 678-81). Amoebae readily consume planktonic bacteria. In addition, they have acquired the ability to eat bacteria within biofilms because amoebae thrive within biologically complex and environmentally harsh soil bio-webs (Rodríguez S, Bishop P, (2007) Three-dimensional quantification of soil biofilms using image analysis. Environ Eng Sci. 24(2): 96-103).

The existence of soil Dictyostelids has been known for almost one hundred and fifty years (Brefeld O. (1869) Abh. Seckenberg Naturforsch. Ges. 7: 85-107). But it was not until 1965, when Cavender and Raper (Cavender J C, Raper K B. (1965) Am J Bot 52: 294-6) developed a quantitative method for their enumeration, that extensive ecological studies of these organisms were undertaken. For the best-characterized genus, Dictyostelium, nine species were found to be common inhabitants of the upper soil and leaf litter layers in the forests of North America (Cavender J C, Raper K B. (1965) The Acrasieae in nature. I. Isolation. Am J Bot 52: 294-6). Since the publication of these early studies, it has been shown that the Dictyostelids occur worldwide in a variety of soil environments (Swanson A, Vadell E, Cavender J. (2001) Global distribution of forest soil dictyostelids J Biogeo 26(1): 133-48). Collectively, the ecological studies suggest that amoebae are truly cosmopolitan both with regard to their geographic distribution and ecological niches.

In some embodiments, D. discoideum isolates are utilized. Strains of amoebae have been isolated that grow on bacteria and on synthetic media (Sussman M, 1966. Biochemical and genetic methods in the study of cellular slime mold development. pp. 397-410. In: Methods in Cell Physiology, Vol. 2, Edited by D Prescott. Academic. Press, New York). High numbers of organisms are easily obtained; Chemical and transposon mutagenesis is routinely used with amoebae to isolate growth and functional mutants (Liwerant I J & Pereira da Silva L H. 1975. Mutat Res. 33(2-3):135-46); Barclay S L & Meller E 1983. Mol Cell Biol. 3:2117-2130). D. discoideum is a haploid easing the genetic characterization of mutant organisms; the genome sequence of D. discoideum has been determined and published (Eichinger L, et al. 2005. Nature. 435:43-57). Also, that genome was compared to the genome of D. purpureum (R. Sucgang et al., 2011, Genome Biology 2011, 12).

Advantageous phenotypes can be linked to multiple genetic mutations, and these mutations can be serially selected using multiple rounds of MNNG mutagenesis. Most questions concerning amoebic treatment can be addressed by manipulating of the amoeba's genome. Amoeba can be genetically altered by chemical mutagenesis or with molecular techniques. For example, D. discoideum is a haploid organism and mutants are easily generated by chemical mutagenesis, gene replacement technologies, and by RNA interference (Barclay S L & Meller E. 1983. Mol Cell Biol. 3:2117-2130; Eichinger L, et al. 2005. Nature. 435:43-57).

In some embodiments, amoebae are stored and/or transported in the spore stage of the life cycle. D. discoideum forms easily germinated temperature-, environment-, and temporally-stable spores. In the absence of a bacterial food supply, essential amino acids become limiting, and D. discoideum sporulates. Spores have been shown to remain viable, without refrigeration, for over 70 years when lyophilized, for shorter times they can be stored in silica gel. When nutrients are available, spores germinate in 6-10 hours to produce amoebae. Spores can be exploited as a means of transport and storage of amoebae used in agricultural and industrial treatments.

In some embodiments, the present disclosure provides kits and/or compositions comprising amoebae. In some embodiments, amoebae are in a form (e.g., spores) that, as noted, is stable for long term storage. In other embodiments, amoebae are stored and transported in different stages. In some embodiments, compositions comprise additional components (e.g., storage reagents, buffers, preservatives, stabilizers, etc.). In some embodiments, amoeba or spores are stored or transported at 80° C. in 10% Dimethyl sulfoxide (DMSO) or 10% glycerol, in the MS2 medium comprising the following: peptone 10 g, dextrose 10 g, Na₂HPO₄×12 H₂O 1 g, KH₂PO₄ 1.5 g, MgSO₄ 0.5 g, per 1 L, 1 g yeast extract (Raper 1984). Another method of long-term storage of spores is lyophilization. In other embodiments, amoebae or spores are stored short-term at 4° C. in medium MS2 solidified with 10 g of agar per L.

In some embodiments, the present disclosure also provides preparations for treating microbial infections and contaminations of plants. Such formulations include dips, sprays, seed dressings, stem injections, sprays, and mists.

In some embodiments, compositions for use in killing microorganisms utilize two or more distinct species of amoebae. Some species of amoebae use different chemo-attractants while other species use the same attractants. For example, for D. mucoroides it is cyclic AMP, while that of P. violaceum is a dipeptide called glorin. This means that when the aggregation centers are first formed, each species is producing its own attractant and will attract only the amoebae that respond to it; they will have no interest in the attractant of the other species and therefore no possibility of commingling.

In another case, Raper and Thom chose two species that had the same chemoattractant, which is cyclic AMP (Raper, K. B., and C. Thorn (1941) Am. J Botany 28: 69-78). Strains were D. mucoroides with white sori and D. purpureum with purple sori. The authors found that these two species co-aggregated into common centers, but there was a surprising sequel. Fruiting bodies arose from the same mound and their sorocarps were either white or purple: the amoebae had separated into two groups in the mound, and the resulting fruiting bodies were pure and all their amoebae were of either one species or the other.

Yet in another case, H. Hagiwara (Hagiwara, H. (1989) The taxonomic study of Japanese Dictyostelid cellular slime molds. Tokyo: National Science Museum Press) discovered a strain of P. pallidum that produces a substance that destroys many other strains of P. pallidum as well as a common wild-type strain D. discoideum. They do so by secreting a lethal molecule that devastates the amoebae of the susceptible victim.

Thus, in some embodiments, two or more compatible species are utilized in a composition. Such combinations are contemplated to find particular use in the killing of drug resistant microorganisms and mixed populations of microorganisms.

In some embodiments, one or more amoebae are administered in combination with known anti-microbial agents. For example, in some embodiments, the agents are pesticides, fungicides, herbicides, fertilizers, or insecticides.

In some embodiments, the fungicide is Regalia® biofungicide. Additional examples of fungicides include, but are not limited to aliphatic nitrogen fungicides (e.g., butylamine, cymoxanil, dodicin, dodine, guazatine, iminoctadine); amide fungicides (e.g., carpropamid, chloraniformethan, cyflufenamid, diclocymet, ethaboxam, fenoxanil, flumetover, furametpyr, isopyrazam, mandipropamid, penthiopyrad, prochloraz, quinazamid, silthiofam, triforine, xiwojunan); acylamino acid fungicides (e.g., benalaxyl (e.g., benalaxyl-M), furalaxyl, metalaxyl (e.g., metalaxyl-M), pefurazoate, valifenalate); anilide fungicides (e.g., benalaxyl (e.g., benalaxyl-M), bixafen, boscalid, carboxin, fenhexamid, fluxapyroxad, isotianil, metalaxyl (e.g., metalaxyl-M), metsulfovax, ofurace, oxadixyl, oxycarboxin, penflufen, pyracarbolid, sedaxane, thifluzamide, tiadinil, vangard); benzanilide fungicides (e.g., benodanil, flutolanil, mebenil, mepronil, salicylanilide, tecloftalam); furanilide fungicides (e.g., fenfuram, furalaxyl, furcarbanil, methfuroxam); sulfonanilide fungicides (e.g., flusulfamide); benzamide fungicides (e.g., benzohydroxamic acid, fluopicolide, fluopyram, tioxymid, trichlamide, zarilamid, zoxamide); furamide fungicides (e.g., cyclafuramid, furmecyclox); phenylsulfamide fungicides (e.g., dichlofluanid, tolylfluanid); sulfonamide fungicides (e.g., amisulbrom, cyazofamid); valinamide fungicides (e.g., benthiavalicarb, iprovalicarb); antibiotic fungicides (e.g., aureofungin, blasticidin-S, cycloheximide, griseofulvin, kasugamycin, moroxydine, natamycin, polyoxins, polyoxorim, streptomycin, validamycin); aromatic fungicides (e.g., biphenyl, chlorodinitronaphthalenes, chloroneb, chlorothalonil, cresol, dicloran, hexachlorobenzene, pentachlorophenol, quintozene, sodium pentachlorophenoxide, tecnazene); arsenical fungicides (e.g., asomate, urbacide); aryl phenyl ketone fungicides (e.g., metrafenone, pyriofenone); benzimidazole fungicides (e.g., benomyl carbendazim, chlorfenazole, cypendazole, debacarb, fuberidazole, mecarbinzid, rabenzazole, thiabendazole); benzimidazole precursor fungicides (e.g., furophanate, thiophanate, thiophanate-methyl); benzothiazole fungicides (e.g., bentaluron, benthiavalicarb, benthiazole, chlobenthiazone, probenazole); botanical fungicides (e.g., allicin, berberine, carvacrol, carvone, osthol); bridged diphenyl fungicides (e.g., bithionol, dichlorophen, diphenylamine, hexachlorophene, parinol); carbamate fungicides (e.g., benthiavalicarb, furophanate, iprovalicarb, propamocarb, pyribencarb, thiophanate, thiophanate-methyl); benzimidazolylcarbamate fungicides (e.g., benomyl, carbendazim, cypendazole, debacarb, mecarbinzid); carbanilate fungicides (e.g., diethofencarb, lvdingjunzhi, pyraclostrobin, pyrametostrobin); conazole fungicides (e.g., conazole fungicides (imidazoles) (e.g., climbazole, clotrimazole, imazalil, oxpoconazole, prochloraz, triflumizole, see also imidazole fungicides), conazole fungicides (triazoles) (e.g., azaconazole, bromuconazole, cyproconazole, diclobutrazol, difenoconazole, diniconazole (e.g., diniconazole-M), epoxiconazole, etaconazole, fenbuconazole, fluquinconazole, flusilazole, flutriafol, furconazole (e.g., furconazole-cis), hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, uniconazole-P, see also triazole fungicides)); copper fungicides (e.g., Bordeaux mixture, Burgundy mixture, Cheshunt mixture, copper acetate, copper carbonate, basic copper hydroxide, copper naphthenate, copper oleate, copper oxychloride, copper silicate, copper sulfate, copper sulfate, basic, copper zinc chromate, cufraneb, cuprobam, cuprous oxide, mancopper, oxine-copper, saisentong); cyanoacrylate fungicides (e.g., benzamacril, phenamacril); dicarboximide fungicides (e.g., famoxadone, fluoroimide); dichlorophenyl dicarboximide fungicides (e.g., chlozolinate, dichlozoline, iprodione, isovaledione, myclozolin, procymidone, vinclozolin); phthalimide fungicides (e.g., captafol, captan, ditalimfos, folpet, thiochlorfenphim); dinitrophenol fungicides (e.g., binapacryl, dinobuton, dinocap (e.g., dinocap-4, dinocap-6, meptyldinocap), dinocton, dinopenton, dinosulfon, dinoterbon, DNOC); dithiocarbamate fungicides (e.g., amobam, asomate, azithiram, carbamorph, cufraneb, cuprobam, disulfiram, ferbam, metam, nabam, tecoram, thiram, urbacide, ziram); cyclic dithiocarbamate fungicides (e.g., dazomet, etem, milneb); polymeric dithiocarbamate fungicides (e.g., mancopper, mancozeb, maneb, metiram, polycarbamate, propineb, zineb); dithiolane fungicides (e.g., isoprothiolane, saijunmao); fumigant fungicides (e.g., dithioether, methyl bromide); hydrazide fungicides (e.g., benquinox, saijunmao); imidazole fungicides (e.g., cyazofamid, fenamidone, fenapanil, glyodin, iprodione, isovaledione, pefurazoate, triazoxide, see also conazole fungicides (imidazoles)); inorganic fungicides (e.g., potassium azide, potassium thiocyanate, sodium azide, sulfur, see also copper fungicides, see also inorganic mercury fungicides); mercury fungicides (e.g., inorganic mercury fungicides (e.g., mercuric chloride, mercuric oxide, mercurous chloride), organomercury fungicides (e.g., (3-ethoxypropyl)mercury bromide, ethylmercury acetate, ethylmercury bromide, ethylmercury chloride, ethylmercury 2,3-dihydroxypropyl mercaptide, ethylmercury phosphate, N-(ethylmercury)-ptoluenesulphonanilide, hydrargaphen, 2-methoxyethylmercury chloride, methylmercury benzoate, methylmercury dicyandiamide, methylmercury pentachlorophenoxide, 8-phenylmercurioxyquinoline, phenylmercuriurea, phenylmercury acetate, phenylmercury chloride, phenylmercury derivative of pyrocatechol, phenylmercury nitrate, phenylmercury salicylate, thiomersal, tolylmercury acetate)); morpholine fungicides (e.g., aldimorph, benzamorf, carbamorph, dimethomorph, dodemorph, fenpropimorph, flumorph, tridemorph); organophosphorus fungicides (e.g., ampropylfos, ditalimfos, EBP, edifenphos, fosetyl, hexylthiofos, inezin, iprobenfos, izopamfos, phosdiphen, pyrazophos, tolclofos-methyl, triamiphos); organotin fungicides (e.g., decafentin, fentin, tributyltin oxide); oxathiin fungicides (e.g., carboxin, oxycarboxin); oxazole fungicides (e.g., dichlozoline, dingjunezuo, drazoxolon, famoxadone, hymexazol, metazoxolon, myclozolin, oxadixyl, vinclozolin); paclobutrazole; polysulfide fungicides (e.g., barium polysulfide, calcium polysulfide, potassium polysulfide, sodium polysulfide); pyrazole fungicides (e.g., bixafen, fluxapyroxad, furametpyr, isopyrazam, penflufen, penthiopyrad, pyraclostrobin, pyrametostrobin, pyraoxystrobin, rabenzazole, sedaxane); pyridine fungicides (e.g., boscalid, buthiobate, dingjunezuo, dipyrithione, fluazinam, fluopicolide, fluopyram, lvdingjunzhi, parinol, pyribencarb, pyridinitril, pyrifenox, pyroxychlor, pyroxyfur); pyrimidine fungicides (e.g., bupirimate, diflumetorim, dimethirimol, ethirimol, fenarimol, ferimzone, nuarimol, triarimol); anilinopyrimidine fungicides (e.g., cyprodinil, mepanipyrim, pyrimethanil); pyrrole fungicides (e.g., dimetachlone, fenpiclonil, fludioxonil, fluoroimide); quinoline fungicides (e.g., ethoxyquin, halacrinate, 8-hydroxyquinoline sulfate, quinacetol, quinoxyfen, tebufloquin); quinone fungicides (e.g., benquinox, chloranil, dichlone, dithianon); quinoxaline fungicides (e.g., chinomethionat, chlorquinox, thioquinox); thiazole fungicides (e.g., ethaboxam, etridiazole, isotianil, metsulfovax, octhilinone, thiabendazole, thifluzamide); thiazolidine fungicides (e.g., flutianil, thiadifluor); thiocarbamate fungicides (e.g., methasulfocarb, prothiocarb); thiophene fungicides (e.g., ethaboxam, silthiofam); triazine fungicides (e.g., anilazine); triazole fungicides (e.g., amisulbrom, bitertanol, fluotrimazole, huanjunzuo, triazbutil, see also conazole fungicides (triazoles)); triazolopyrimidine fungicides (e.g., ametoctradin); urea fungicides (e.g., bentaluron, pencycuron, quinazamid); unclassified fungicides (e.g., acibenzolar, acypetacs, allyl alcohol, benzalkonium chloride, bethoxazin, bromothalonil, chloropicrin, DBCP, dehydroacetic acid, diclomezine, diethyl pyrocarbonate, ethylicin, fenaminosulf, fenitropan, fenpropidin, formaldehyde, furfural, hexachlorobutadiene, methyl iodide, methyl isothiocyanate, nitrostyrene, nitrothal-isopropyl, OCH, 2-phenylphenol, phthalide, piperalin, propamidine, proquinazid, pyroquilon, sodium orthophenylphenoxide, spiroxamine, sultropen, thicyofen, tricyclazole, zinc naphthenate); or a strobilurin or strobilurin derivative.

Examples of insecticides include, but are not limited to, antibiotic insecticides (e.g., allosamidin, thuringiensis); macrocyclic lactone insecticides (e.g., avermectin insecticides (e.g., abamectin, doramectin, emamectin, eprinomectin, ivermectin, selamectin), milbemycin insecticides (e.g., lepimectin, milbemectin, milbemycin oxime, and moxidectin), spinosyn insecticides (e.g., spinetoram and spinosad)); arsenical insecticides (e.g., calcium arsenate, copper acetoarsenite, copper arsenate, lead arsenate, potassium arsenite, sodium arsenite); botanical insecticides (e.g., allicin, anabasine, azadirachtin, carvacrol, d-limonene, matrine, nicotine, nornicotine, oxymatrine, pyrethrins (e.g., cinerins, (e.g., cinerin I, cinerin II), jasmolin I, jasmolin II, pyrethrin I, pyrethrin II), quassia, rhodojaponin-III, rotenone, ryania, sabadilla, triptolide); carbamate insecticides (e.g., bendiocarb, carbaryl); benzofuranyl methylcarbamate insecticides (e.g., benfuracarb, carbofuran, carbosulfan, decarbofuran, furathiocarb); dimethylcarbamate insecticides (e.g., dimetan, dimetilan, hyquincarb, isolan, pirimicarb, pyramat); oxime carbamate insecticides (e.g., alanycarb, aldicarb, aldoxycarb, butocarboxim, butoxycarboxim, methomyl, nitrilacarb, oxamyl, tazimcarb, thiocarboxime, thiodicarb, thiofanox); phenyl methylcarbamate insecticides (e.g., allyxycarb, aminocarb, bufencarb, butacarb, carbanolate, cloethocarb, CPMC, dicresyl, dimethacarb, dioxacarb, EMPC, ethiofencarb, fenethacarb, fenobucarb, isoprocarb, methiocarb, metolcarb, mexacarbate, promacyl, promecarb, propoxur, trimethacarb, XMC, xylylcarb); desiccant insecticides (e.g., boric acid, diatomaceous earth, silica gel); diamide insecticides (e.g., chlorantraniliprole, cyantraniliprole, flubendiamide); dinitrophenol insecticides (e.g., dinex, dinoprop, dinosam, DNOC); fluorine insecticides (e.g., barium hexafluorosilicate, cryolite, flursulamid, sodium fluoride, sodium hexafluorosilicate, sulfluramid); formamidine insecticides (e.g., amitraz, chlordimeform, formetanate, formparanate, medimeform, semiamitraz); fumigant insecticides (e.g., acrylonitrile, carbon disulfide, carbon tetrachloride, chloroform, chloropicrin, paradichlorobenzene, 1,2-dichloropropane, dithioether, ethyl formate, ethylene dibromide, ethylene dichloride, ethylene oxide, hydrogen cyanide, methyl bromide, methyl iodide, methylchloroform, methylene chloride, naphthalene, phosphine, sulfuryl fluoride, tetrachloroethane); inorganic insecticides (e.g., borax, boric acid, calcium polysulfide, copper oleate, diatomaceous earth, mercurous chloride, potassium thiocyanate, silica gel, sodium thiocyanate, see also arsenical insecticides, see also fluorine insecticides); insect growth regulators (e.g., chitin synthesis inhibitors (e.g., bistrifluron, buprofezin, chlorbenzuron, chlorfluazuron, cyromazine, dichlorbenzuron, diflubenzuron, flucycloxuron, flufenoxuron, hexaflumuron, lufenuron, novaluron, noviflumuron, penfluron, teflubenzuron, triflumuron); juvenile hormone mimics (e.g., dayoutong, epofenonane, fenoxycarb, hydroprene, kinoprene, methoprene, pyriproxyfen, triprene); juvenile hormones (e.g., juvenile hormone I, juvenile hormone II, juvenile hormone III); moulting hormone agonists (e.g., chromafenozide, furan tebufenozide, halofenozide, methoxyfenozide, tebufenozide, yishijing); moulting hormones (e.g., α-ecdysone, ecdysterone); moulting inhibitors (e.g., diofenolan); precocenes (e.g., precocene I, precocene II, precocene III); unclassified insect growth regulators (e.g., dicyclanil)); nereistoxin analogue insecticides (e.g., bensultap, cartap, polythialan, thiocyclam, thiosultap); nicotinoid insecticides (e.g., flonicamid); nitroguanidine insecticides (e.g., clothianidin, dinotefuran, imidacloprid, imidaclothiz, thiamethoxam); nitromethylene insecticides (e.g., nitenpyram, nithiazine); pyridylmethylamine insecticides (e.g., acetamiprid, imidacloprid, nitenpyram, paichongding, thiacloprid); organochlorine insecticides (e.g., bromo-DDT, camphechlor, DDT (e.g., pp′-DDT), ethyl-DDD, HCH (e.g., gamma-HCH, lindane), methoxychlor, pentachlorophenol, TDE); cyclodiene insecticides (e.g., aldrin, bromocyclen, chlorbicyclen, chlordane, chlordecone, dieldrin, dilor, endosulfan (e.g., alpha endosulfan), endrin, HEOD heptachlor, HHDN, isobenzan, isodrin, kelevan, mirex), organophosphorus insecticides (e.g., organophosphate insecticides (e.g., bromfenvinfos, calvinphos, chlorfenvinphos, crotoxyphos, dichlorvos, dicrotophos, dimethylvinphos, fospirate, heptenophos, methocrotophos, mevinphos monocrotophos, naled, naftalofos, phosphamidon, propaphos, TEPP, tetrachlorvinphos); organothiophosphate insecticides (e.g., dioxabenzofos, fosmethilan, phenthoate); aliphatic organothiophosphate insecticides (e.g., acethion, acetophos, amiton, cadusafos, chlorethoxyfos, chlormephos, demephion (e.g., demephion-O, demephion-S), demeton (e.g., demeton-O, demeton-S), demeton methyl (e.g., demeton-O-methyl, demeton-S-methyl), disulfoton, ethion ethoprophos, IPSP, isothioate, malathion, methacrifos, methylacetophos, oxydemetonmethyl, oxydeprofos, oxydisulfoton, phorate, sulfotep, terbufos, thiometon); aliphatic amide organothiophosphate insecticides (e.g., amidithion, cyanthoate, dimethoate, ethoate-methyl, formothion, mecarbam, omethoate, prothoate, sophamide, vamidothion), oxime organothiophosphate insecticides (e.g., chlorphoxim, phoxim, phoxim-methyl); heterocyclic organothiophosphate insecticides (e.g., azamethiphos, colophonate, coumaphos, coumithoate, dioxathion, endothion, menazon, morphothion, phosalone, pyraclofos, pyridaphenthion, quinothion); benzothiopyran organothiophosphate insecticides (e.g., dithicrofos, thicrofos); benzotriazine organothiophosphate insecticides (e.g., azinphos-ethyl, azinphos-methyl); isoindole organothiophosphate insecticides (e.g., dialifos, phosmet); isoxazole organothiophosphate insecticides (e.g., isoxathion, zolaprofos); pyrazolopyrimidine organothiophosphate insecticides (e.g., chlorprazophos, pyrazophos); pyridine organothiophosphate insecticides (e.g., chlorpyrifos, chlorpyrifos-methyl); pyrimidine organothiophosphate insecticides (e.g., butathiofos, diazinon, etrimfos, lirimfos, pirimioxyphos pirimiphos-ethyl, pirimiphos-methyl, primidophos, pyrimitate, tebupirimfos); thiadiazole organothiophosphate insecticides (e.g., athidathion, lythidathion, methidathion, prothidathion); triazole organothiophosphate insecticides (e.g., isazofos, triazophos); phenyl organothiophosphate insecticides (e.g., azothoate, bromophos, bromophos-ethyl, carbophenothion, chlorthiophos, cyanophos, cythioate, dicapthon, dichlofenthion, etaphos, famphur, fenchlorphos, fenitrothion, fensulfothion, fenthion, fenthion-ethyl, heterophos, jodfenphos, mesulfenfos, parathion, parathion-methyl, phenkapton, phosnichlor, profenofos, prothiofos, sulprofos, temephos, trichlormetaphos-3, trifenofos, xiaochongliulin)); phosphonate insecticides (e.g., butonate, trichlorfon); phosphonothioate insecticides (e.g., mecarphon); phenyl ethylphosphonothioate insecticides (e.g., fonofos, trichloronat); phenyl phenylphosphonothioate insecticides (e.g., cyanofenphos, EPN, leptophos); phosphoramidate insecticides (e.g., crufomate, fenamiphos, fosthietan, mephosfolan, phosfolan, phosfolan-methyl pirimetaphos); phosphoramidothioate insecticides (e.g., dimefox, mazidox, mipafox, schradan); oxadiazine insecticides (e.g., indoxacarb); oxadiazolone insecticides (e.g., metoxadiazone); phthalimide insecticides (e.g., dialifos, phosmet, tetramethrin); pyrazole insecticides (e.g., chlorantraniliprole, cyantraniliprole, dimetilan, isolan, tebufenpyrad, tolfenpyrad); phenylpyrazole insecticides (e.g., acetoprole, ethiprole, fipronil, pyraclofos, pyrafluprole, pyriprole, vaniliprole); pyrethroid insecticides (e.g., pyrethroid ester insecticides (e.g., acrinathrin, allethrin (e.g., bioallethrin, esdépalléthrine), barthrin, bifenthrin, bioethanomethrin brofenvalerate, brofluthrinate, bromethrin, butethrin, chlorempenthrin, cyclethrin, cycloprothrin cyfluthrin (e.g., beta-cyfluthrin), cyhalothrin (e.g., gamma-cyhalothrin, lambda-cyhalothrin), cypermethrin (e.g., alpha-cypermethrin, beta-cypermethrin, theta-cypermethrin, zeta-cypermethrin), cyphenothrin, deltamethrin, dimefluthrin, dimethrin, empenthrin, d-fanshiluquebingjuzhi, fenfluthrin, fenpirithrin, fenpropathrin, fenvalerate (e.g., esfenvalerate), flucythrinate, fluvalinate (e.g., tau fluvalinate), furamethrin, furethrin, imiprothrin, japothrins, kadethrin, meperfluthrin, methothrin, metofluthrin, pentmethrin, permethrin (e.g., biopermethrin, transpermethrin), phenothrin, prallethrin, profluthrin, proparthrin, pyresmethrin, resmethrin (e.g., bioresmethrin, cismethrin), tefluthrin, terallethrin, tetramethrin, tetramethylfluthrin, tralocythrin, tralomethrin, transfluthrin, valerate; pyrethroid ether insecticides (e.g., etofenprox, flufenprox, halfenprox, protrifenbute, silafluofen); pyrethroid oxime insecticides (e.g., sulfoxime, thiofluoximate)); pyrimidinamine insecticides (e.g., flufenerim, pyrimidifen); pyrrole insecticides (e.g., chlorfenapyr); tetramic acid insecticides (e.g., spirotetramat); tetronic acid insecticides (e.g., spiromesifen); thiazole insecticides (e.g., clothianidin, imidaclothiz, thiamethoxam, thiapronil); thiazolidine insecticides (e.g., tazimcarb, thiacloprid); thiourea insecticides (e.g., diafenthiuron); urea insecticides (e.g., flucofuron, sulcofuron, see also chitin synthesis inhibitors); unclassified insecticides (e.g., closantel, copper naphthenate, crotamiton EXD, fenazaflor, fenoxacrim, hydramethylnon, isoprothiolane malonoben, metaflumizone, nifluridide, plifenate, pyridaben, pyridalyl, pyrifluquinazon, rafoxanide, sulfoxaflor, triarathene, triazamate).

Examples of pesticides include, but are not limited to, acaricides, avicides, chemosterilants, herbicides, insecticides, molluscicides, plant growth regulators, virucides, algicides, bactericides, fungicides, insect attractants, mammal repellents, nematicides, rodenticides, antifeedants, bird repellents, herbicide safeners, insect repellents, mating disrupters, plant activators, synergists, chemical classes, and miscellaneous.

The present disclosure is not limited to a particular herbicide. Examples include, but are not limited to, amide herbicides (e.g., allidochlor, amicarbazone, beflubutamid, benzadox, benzipram, bromobutide, cafenstrole, CDEA, cyprazole, dimethenamid (e.g., dimethenamid-P), diphenamid, epronaz, etnipromid, fentrazamide, flucarbazone, flupoxam, fomesafen, halosafen, huangcaoling, isocarbamid, isoxaben, napropamide, naptalam, pethoxamid, propyzamide, quinonamid, saflufenacil, tebutam); anilide herbicides (e.g., chloranocryl, cisanilide, clomeprop, cypromid, diflufenican, erlujixiancaoan, etobenzanid, fenasulam, flufenacet, flufenican, ipfencarbazone, mefenacet, mefluidide, metamifop, monalide, naproanilide, pentanochlor, picolinafen, propanil, sulfentrazone); arylalanine herbicides (e.g., benzoylprop, flamprop (e.g., flamprop-M)); chloroacetanilide herbicides (e.g., acetochlor, alachlor, butachlor, butenachlor, delachlor, diethatyl, dimethachlor, ethachlor, ethaprochlor, metazachlor, metolachlor (e.g., S-metolachlor), pretilachlor, propachlor, propisochlor, prynachlor, terbuchlor, thenylchlor, xylachlor); sulfonanilide herbicides (e.g., benzofluor, cloransulam, diclosulam, florasulam, flumetsulam, metosulam, perfluidone, pyrimisulfan, profluazol); sulfonamide herbicides (e.g., asulam, carbasulam, fenasulam, oryzalin, penoxsulam, pyroxsulam, see also sulfonylurea herbicides); thioamide herbicides (e.g., bencarbazone, chlorthiamid); antibiotic herbicides (e.g., bilanafos); aromatic acid herbicides (e.g., benzoic acid herbicides (e.g., chloramben, dicamba, 2,3,6-TBA, tricamba); pyrimidinyloxybenzoic acid herbicides (e.g., bispyribac, pyriminobac); pyrimidinylthiobenzoic acid herbicides (e.g., pyrithiobac); phthalic acid herbicides (e.g., chlorthal); picolinic acid herbicides (e.g., aminopyralid, clopyralid, picloram); quinolinecarboxylic acid herbicides (e.g., quinclorac, quinmerac)); arsenical herbicides (e.g., cacodylic acid, CMA, DSMA, hexaflurate, MAA, MAMA, MSMA, potassium arsenite, sodium arsenite); benzoylcyclohexanedione herbicides (e.g., ketospiradox, mesotrione, sulcotrione, tefuryltrione, tembotrione); benzofuranyl alkylsulfonate herbicides (e.g., benfuresate, ethofumesate); benzothiazole herbicides (e.g., benazolin, benzthiazuron, fenthiaprop, mefenacet, methabenzthiazuron); carbamate herbicides (e.g., asulam, carboxazole, chlorprocarb, dichlormate, fenasulam, karbutilate, terbucarb); carbanilate herbicides (e.g., barban, BCPC, carbasulam, carbetamide, CEPC, chlorbufam, chlorpropham, CPPC, desmedipham, phenisopham, phenmedipham, phenmedipham-ethyl, propham, swep); cyclohexane oxime herbicides (e.g., alloxydim, butroxydim, clethodim, cloproxydim, cycloxydim, profoxydim, sethoxydim, tepraloxydim, tralkoxydim); cyclopropylisoxazole herbicides (e.g., isoxachlortole, isoxaflutole); dicarboximide herbicides (e.g., cinidon-ethyl, flumezin, flumiclorac, flumioxazin, flumipropyn, see also uracil herbicides); dinitroaniline herbicides (e.g., benfluralin, butralin, chlornidine, dinitramine, dipropalin, ethalfluralin, fluchloralin, isopropalin, methalpropalin, nitralin, oryzalin, pendimethalin, prodiamine, profluralin, trifluralin); dinitrophenol herbicides (e.g., dinofenate, dinoprop, dinosam, dinoseb, dinoterb, DNOC, etinofen, medinoterb); diphenyl ether herbicides (e.g., ethoxyfen); nitrophenyl ether herbicides (e.g., acifluorfen, aclonifen, bifenox, chlomethoxyfen, chlornitrofen, etnipromid, fluorodifen, fluoroglycofen, fluoronitrofen, fomesafen, fucaomi, furyloxyfen, halosafen, lactofen, nitrofen, nitrofluorfen, oxyfluorfen); dithiocarbamate herbicides (e.g., dazomet, metam); halogenated aliphatic herbicides (e.g., alorac, chloropon, dalapon, flupropanate, hexachloroacetone, methyl bromide, methyl iodide, monochloroacetic acid, SMA, TCA); imidazolinone herbicides (e.g., imazamethabenz, imazamox, imazapic, imazapyr, imazaquin, imazethapyr); inorganic herbicides (e.g., ammonium sulfamate, borax, calcium chlorate, copper sulfate, ferrous sulfate, potassium azide, potassium cyanate, sodium azide, sodium chlorate, sulfuric acid); nitrile herbicides (e.g., bromobonil, bromoxynil, chloroxynil, dichlobenil, iodobonil, ioxynil, pyraclonil); organophosphorus herbicides (e.g., amiprofos-methyl, amiprophos, anilofos, bensulide, bilanafos, butamifos, 2,4-DEP, DMPA, EBEP, fosamine, glufosinate (e.g., glufosinate-P, glyphosate, huangcaoling piperophos); oxadiazolone herbicides (e.g., dimefuron, methazole, oxadiargyl, oxadiazon); oxazole herbicides (e.g., carboxazole, fenoxasulfone, isouron, isoxaben, isoxachlortole, isoxaflutole, methiozolin, monisouron, pyroxasulfone, topramezone); phenoxy herbicides (e.g., bromofenoxim, clomeprop, 2,4-DEB, 2,4-DEP, difenopenten, disul, erbon, etnipromid, fenteracol, trifopsime); phenoxyacetic herbicides (e.g., 4-CPA, 2,4-D, 3,4-DA, MCPA, MCPA-thioethyl, 2,4,5-T); phenoxybutyric herbicides (e.g., 4-CPB, 2,4-DB, 3,4-DB, MCPB, 2,4,5-TB); phenoxybutyric herbicides (e.g., 4-CPB, 2,4-DB, 3,4-DB, MCPB, 2,4,5-TB); phenoxypropionic herbicides (e.g., cloprop, 4-CPP, dichlorprop (e.g., dichlorprop-P), 3,4-DP, fenoprop, mecoprop (e.g., mecoprop-P); aryloxyphenoxypropionic herbicides (e.g., chlorazifop, clodinafop, clofop, cyhalofop, diclofop, fenoxaprop (e.g., fenoxaprop-P); fenthiaprop, fluazifop (e.g., fluazifop-P), haloxyfop (e.g., haloxyfop-P), isoxapyrifop, metamifop, propaquizafop, quizalofop (e.g., quizalofop-P), trifop); phenylenediamine herbicides (e.g., dinitramine, prodiamine); pyrazole herbicides (e.g., azimsulfuron, difenzoquat, halosulfuron, metazachlor, metazosulfuron, pyrazosulfuron, pyroxasulfone); pyrazole herbicides (e.g., benzofenap, pyrasulfotole, pyrazolynate, pyrazoxyfen, topramezone); phenylpyrazole herbicides (e.g., fluazolate, nipyraclofen, pinoxaden, pyraflufen); pyridazine herbicides (e.g., credazine, pyridafol, pyridate); pyridazinone herbicides (e.g., brompyrazon, chloridazon, dimidazon, flufenpyr, metflurazon, norflurazon, oxapyrazon, pydanon); pyridine herbicides (e.g., aminopyralid, cliodinate, clopyralid, diflufenican, dithiopyr, flufenican, fluroxypyr, haloxydine picloram, picolinafen, pyriclor, pyroxsulam, thiazopyr, triclopyr); pyrimidinediamine herbicides (e.g., iprymidam, tioclorim); pyrimidinyloxybenzylamine herbicides (e.g., pyribambenz-isopropyl, pyribambenzpropyl); quaternary ammonium herbicides (e.g., cyperquat, diethamquat, difenzoquat, diquat, morfamquat, paraquat); thiocarbamate herbicides (e.g., butylate, cycloate, diallate, EPTC, esprocarb, ethiolate, isopolinate, methiobencarb, molinate, orbencarb, pebulate, prosulfocarb, pyributicarb, sulfallate, thiobencarb, tiocarbazil, tri-allate, vernolate); thiocarbonate herbicides (e.g., dimexano, EXD, proxan); thiourea herbicides (e.g., methiuron); triazine herbicides (e.g., dipropetryn, fucaojing, trihydroxytriazine); chlorotriazine herbicides (e.g., atrazine, chlorazine, cyanazine, cyprazine, eglinazine, ipazine, mesoprazine, procyazine, proglinazine, propazine, sebuthylazine, simazine, terbuthylazine, trietazine); fluoroalkyltriazine herbicides (e.g., indaziflam, triaziflam); methoxytriazine herbicides (e.g., atraton, methometon, prometon, secbumeton, simeton, terbumeton); methylthiotriazine herbicides (e.g., ametryn, aziprotryne, cyanatryn, desmetryn, dimethametryn, methoprotryne, prometryn, simetryn, terbutryn); triazinone herbicides (e.g., ametridione, amibuzin, ethiozin, hexazinone, isomethiozin, metamitron, metribuzin); triazole herbicides (e.g., amitrole, cafenstrole, epronaz, flupoxam); triazolone herbicides (e.g., amicarbazone, bencarbazone, carfentrazone, flucarbazone, ipfencarbazone, propoxycarbazone, sulfentrazone, thiencarbazone); triazolopyrimidine herbicides (e.g., cloransulam, diclosulam, florasulam, flumetsulam, metosulam, penoxsulam, pyroxsulam); uracil herbicides (e.g., benzfendizone, bromacil, butafenacil, flupropacil, isocil, lenacil, saflufenacil, terbacil); urea herbicides (e.g., benzthiazuron, cumyluron, cycluron, dichloralurea, diflufenzopyr, isonoruron, isouron, methabenzthiazuron, monisouron, noruron), phenylurea herbicides (e.g., anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluron, phenobenzuron, siduron, tetrafluron, thidiazuron); sulfonylurea herbicides (e.g., pyrimidinylsulfonylurea herbicides (e.g., amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, metazosulfuron, methiopyrisulfuron, nicosulfuron, orthosulfamuron, oxasulfuron, primisulfuron, propyrisulfuron, pyrazosulfuron, rimsulfuron, sulfometuron, sulfosulfuron, trifloxysulfuron), triazinylsulfonylurea herbicides (e.g., chlorsulfuron, cinosulfuron, ethametsulfuron, iodosulfuron, metsulfuron, prosulfuron, thifensulfuron, triasulfuron, tribenuron, triflusulfuron, tritosulfuron)); thiadiazolylurea herbicides (e.g., buthiuron, ethidimuron, tebuthiuron, thiazafluron, thidiazuron); unclassified herbicides e.g., acrolein, allyl alcohol, aminocyclopyrachlor, azafenidin, bentazone, bentranil, benzobicyclon, bicyclopyrone, buthidazole, calcium cyanamide, cambendichlor, chlorfenac, chlorfenprop, chlorflurazole, chlorflurenol, cinmethylin, clomazone, CPMF, cresol, cyanamide, ortho-dichlorobenzene, dimepiperate, dithioether, endothal, fluoromidine, fluridone, flurochloridone, flurtamone, fluthiacet, indanofan, methoxyphenone, methyl isothiocyanate, OCH, oxaziclomefone, pelargonic acid, pentachlorophenol, pentoxazone, phenylmercury acetate, prosulfalin, pyribenzoxim, pyriftalid, quinoclamine, rhodethanil, sulglycapin, thidiazimin, tridiphane, trimeturon, tripropindan, tritac).

Fertilizers include any micronutrient/fertilizer containing formulations that meet the definition of fertilizer as given by the AAPFCO (American Association of Plant Food Control Officials). Fertilizers include any material, whether of natural or synthetic origin, that is applied to soils or to plant tissues to supply one or more plant nutrients. Fertilizers include single nutrient (“straight”) fertilizers (e.g., ammonium nitrate, superphosphates, etc.) as well as multinutrient (e.g., binary (NP, NK, PK) fertilizers and NPK fertilizers) fertilizers (e.g., monoammonium phosphate, diammonium phosphate, etc.). Nutrients include but are not limited to nitrogen, phosphorus, potassium, calcium, magnesium, sulfur, copper, iron, manganese, molybdenum, zinc, boron, silicon, cobalt, and vanadium.

### EXPERIMENTAL

The following examples are provided in order to demonstrate and further illustrate certain preferred embodiments and aspects of the present disclosure and are not to be construed as limiting the scope thereof.

### Example 1

### Methods

### Application Timing:

3 applications per treatment, Mid-Bloom (50-70%), Full Bloom, Petal Fall

### Treatment Prep:

All treatments are labeled with an ID # and provided as dried pellets in a 50 ml conical tube. These tubes are vacuum sealed in plastic bags and clearly grouped, labeled, and organized. Each tube contains sufficient spores to make up 7.6 Liters (2 gallons) of treatment solution. The contents of each tube are dissolved in ˜25 mls of water with 0.05% (1:2000) Silwet L77 (Momentive, Waterford, N.Y.) by vortexing for 20 seconds (or shaking vigorously if no vortex is available), followed by incubation at room temp for 15 min and then vortexing again for 30 seconds. The solution becomes very cloudy after vortexing. Some small (1-2 mm) particles may not fully dissolve and this is acceptable. If larger particles remain (unlikely), further incubation and vortexing is performed.

Once dissolved, the contents are added to 2 gallons of water containing 0.05% Silwet L77 for application. Treatments is preferably applied as soon as possible but anytime within 4 hours is acceptable. The spores begin to settle out of solution rather quickly so the sprayer contents should be mixed/shaken just before spraying each tree and every 5 minutes during spraying.

Some treatments include combinations of multiple Dicty strains. These are clearly labeled (ex “2-4 combo”) and multiple tubes are contained within the smallest vacuum bags. The pellets are resuspended and combined in the sprayer.

### Evaluation:

Materials for FB control is evaluated in a in a 17-yr-old ‘Gala’ apple orchard located at the Oregon State University, Botany & Plant Pathology Field Laboratory near Corvallis, Oreg. Experiments are arranged in a randomized, complete block design with 4 replications of ½ tree plots. Flower cluster density on individual trees (counted prebloom) and tree location is considered in the assignment of trees to blocks in the plot design.

Amoebae treatments are compared to a water-treated control, and to treatments of FireLine (oxytetracycline hydrochloride 17% a.i, AgroSource Inc., Cranford, N.J.) and FireWall (streptomycin sulfate 17% a.i, AgroSource), which represent industrial standards.

Treatments are applied during early morning at the following bloom stages: 70% bloom, full bloom, and petal fall. Treatment suspensions are sprayed to near runoff with backpack sprayers equipped with hand wands (1.5 liters per half tree). At full bloom, a motorized 25-gallon tank sprayer equipped with a hand wand is used to fog a suspension of freeze-dried cells of Ea strain 153N (nalidixic acid-resistant (50 μg/ml), streptomycin and oxytetracycline sensitive pathogen strain), which is prepared at 5×10⁵ CFU per ml (2 liters per tree).

Five flower clusters (˜6 flowers cluster) are sampled from each replicate ½ tree on each of three dates: full bloom, petal fall, and one-week post petal fall. Each flower cluster sample is immersed and massaged in 30 ml of sterile phosphate buffer followed by sonication for 3 minutes. After sonication, a 10-μl sample of the flower wash and two 1:100 dilutions will be spread on CCT medium amended with nalidixic acid (50 μg/ml) to selectively enumerate Ea153N. Log₁₀-transformed population data will be analyzed by ANOVA.

Incidence of FB is determined by counting the number of blighted flower clusters (i.e. strikes) on each tree during inspections in the month of May. Blighted clusters are removed immediately after counting. Total number of blighted flower clusters per tree (strikes) and incidence of disease (total strikes/total number of clusters per tree) is subjected to analysis of variance).

### Protocol for Harvesting Dicty Spores:

Spores are grown on flat sheets. Once a sheet is fully mature, 37.5 ml of DB-Sil is added around the plate. Using silicone spatula, liquid and stems are scraped to one short side of the sheet. All liquid and stems are moved to the opposite corner. Stems are disturbed with a spatula to release spores. While the sheet is elevated, stems are washed up the sheet to let liquid run down into corner. Once all stems are removed, they are scraped to one side and squeezed with a spatula. Liquid is transferred to a cell strainer (100 μm) over a 50 ml tube labeled for the strain. Plates are washed as above with 20-25 mls (depending on space left in 50 ml tube). The stem pellet is resuspended and washed again as above. Liquid is transferred to 50 ml tube again and stems are squeezed in a strainer with a pipette to remove liquid. The stem ball is removed from the tray. The lid is replaced on the tray and labeled.

### Protocol for Antibiotic Treating and Vacuum Drying Spores:

Spores are centrifuged at 1,500 G for 5 min, the supernatant is decanted and resuspended in 15 ml DB+ with 200 μg/ml Streptomycin and 17 μg/ml Chloramphenicol. The spores are incubated at 29 degrees C. with shaking at 300 RPM for 2 hours. Centrifugation is repeated and the pellet is resuspended in 15 ml DB-Sil and the 5 tubes are combined to 2 tubes. All pellets are spun and resuspended in 40-50 ml (total volume). Ten microliters from the tube are diluted 100-1000 fold in DB-Sil and counted using hemocytometer. Volume for 7.6E9 spores is aliquoted into 50 ml tubes (as many as necessary) and the remaining stored as in tube labeled # “p” (to indicate partial tube) and indicating the number of spores in this tube. The spores are spun and resuspended in 5 mls of 10% nonfat dry milk (in sterile water). The supernatant is decanted, tubes are transferred deep into aluminum beads in vacuum chamber, and the lid is loosened to nearly off. Tubes are vacuumed to −29 inHg and the valve is closed. Tubes are left under vacuum for at least 16 hours or until visible tube is completely dry (no whitish color in the center of the pellet). Vacuum is released slowly and tubes are immediately capped tightly and placed in fridge.

Screen Dicty Isolates In Vitro for Attributes Needed for Biocontrol in Orchards and for Decontaminating Plants

One or more strains of Dictyostelids (Dicty, FIG. 1) were investigated as a biological treatment for bacterial infections of plants, with a focus on pome fruit orchards. Specifically, a treatment for the bacterial pathogen Ea that causes FB^{2,3} and Clavibacter michiganensis subsp. michiganensis (Cmm) that causes bacterial canker in tomatoes⁵ were developed.

44 Dicty strains able to reduce Ea Colony Forming Units (CFU)/ml in vitro by >99.95% were identified, and all 44 able to reduce the Cmm CFU/ml by >99.95% in vitro. After identifying the top 10 strains active against Ea, two field trials were conducted on apples in 2017, including one combination treatment and one dose response.

Dicty strains were tested for their ability to feed on Ea as a prey source and produce spores. It was determined that 96 of 101 (95%) strains could both feed and produce spores on Ea. At three different temperatures (20°, 25°, and 30° C.), the 96 Dicty strains able to feed on Ea for were tested for their degree of bacterial clearing. It was found that 44 of the 96 (45.8%) Dicty strains that fed on Ea were able to reduce the Ea CFU/ml by >99.95% compared to the control Ea lawn at 1 of the 3 temperatures tested. Because biofilms are major contributors to bacterial pathogenesis in agriculture⁸, the ability of top Dicty to also feed on biofilms of Ea was determined. Testing top performers, it was found that 31/44 (70.5%) Dicty strains were able to reduce the Ea CFU/ml of biofilms by >99.95% compared to a control Ea at 1 of 3 temperatures (20°, 25°, and 30° C.). The 44 Dicty strains identified were tested for their feeding specificity to compare pathogenic and non-pathogenic bacterial species found on plant surfaces including: 10 known commensals, 11 unknown commensals from leaves, and 4 unknown commensals from blossoms. From this screen, those Dicty strains that do not selectively consume pathogenic bacterial species were excluded. 18/31 (58%) Dicty strains tested selectively reduced pathogenic Ea over at least 15/25 (60%) commensal bacteria. The top 31 strains from above were tested with common copper- and sulfur-based products used in the field to control FB and other plant disease^{7,8,9}. The products were tested in concurrent and consecutive applications to determine if the chemicals affected Dicty germination and feeding, respectively. 8/31 (26%) and 19/31 (61%) Dicty strains tested were resistant to copper octanoate when applied concurrently and consecutively, respectively. 13/31 (42%) and 23/31 (74%) Dicty strains tested were resistant to sulfur when applied concurrently and consecutively, respectively.

Test Most Robust Biofilm Eaters In-Planta

Importantly, Ea biofilms develop in vasculature of apple and pear trees and block flow of nutrients. ^{7,8,9,10,11,12}. Biofilms are communities of bacteria from which planktonic bacteria are gradually released to quickly multiply, and by doing so do serious harm to the plant. Dicty strains able to reduce biofilm-enmeshed pathogen in-planta were identified.

In planta-experiments were performed on apple seedlings in a growth chamber which provided control of temperature, light duration, and humidity to mimic field conditions. This model system provided the ability to complete experiments in winter months prior to spring field trials allowing one to use this data to better prioritize Dicty strains for field trials. More than 50 pairwise combinations were tested in vitro to identify combinations without negative interactions. Field trials were conducted to assess the ability of select Dicty strains and combinations to reduce blossom blight.

Two strains that reduced blossom blight in a field trial conducted in Oregon were identified. The top-performing strain was as effective as antibiotics.

Blossom assay: One Dicty strain (WS-20) significantly reduced the average length of shoot necrosis compared to the Ea infected control (*p<0.05).

Wounded leaf assay: One Dicty strain (WS-28) completely prevented FB disease (FIG. 2a). Dicty complete their life cycle on apple leaves as seen by formation of sporangia on leaves treated with Dicty, indicating that Dicty treatment on leaves is promising because Dicty can undergo their developmental cycle on plant surfaces and didn't harm leaves (FIG. 2b).

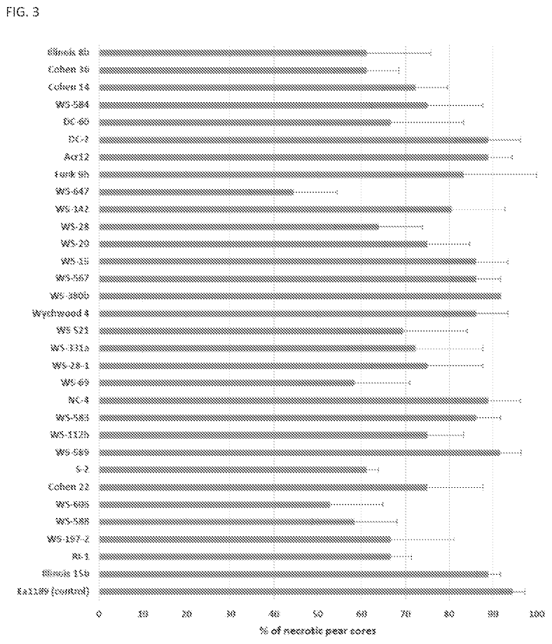

Pear fruit assay: 11 Dicty strains which were efficacious in preventing necrosis in a biocontrol predictive pear core assay were identified (FIG. 3).

Compared to control, 20 of 31 tested Dicty strains (64.5%) reduced disease severity by ≥75% on apple leaves, or bacterial CFU/ml by >0.5 log10, or both compared to the control.

Blossom Blight: The top 31 Dicty strains were tested on detached blossoms from Gala trees (from an orchard) in vitro (Table 1). 17/31 (54.8%) Dicty strains reduced disease severity by >50% and/or the bacterial CFU/ml by >0.5 log 10, compared to the Ea control (Table 1).

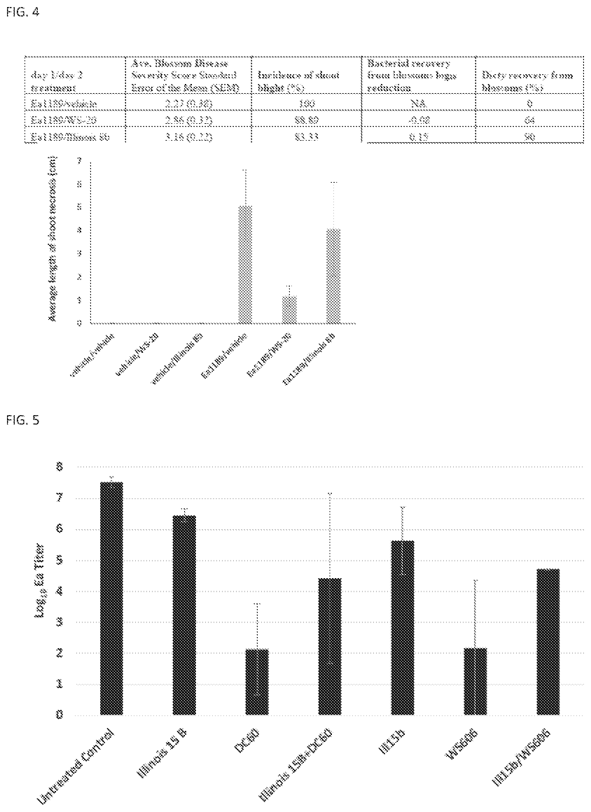

Growth Chamber: 13 of 23 Dicty strains tested (57%) significantly reduced disease severity from grade 3-4 to grade 1 in apple seedlings (FIG. 4).

Ten individual Dicty strains were tested in 13 different treatments in external field trials, based on results from all above related to Ea. A total of 13 Dicty treatments including 10 individual Dicty strains, 1 combination of strains, and a dose series for one strain in two external field trials were conducted in Oregon. Additional experimentation helped exclude 8/31 strains based on their potential ability to facilitate the transmission of bacteria (so called farmers” (Brock, D. A., et al. (2013). “Social amoeba farmers carry defensive symbionts to protect and privatize their crops.” Nature communications 4: 2385). Dicty did not cause blemishing or deformity to fruit or vegetative tissue of trees in these trials. While all treatments reduced blighted floral clusters, one strain showed particularly strong (82%) disease control which was statistically identical to antibiotic treatment (FIG. 5) and another provided moderate, reproducible disease control, but at levels shy of statistical significance.

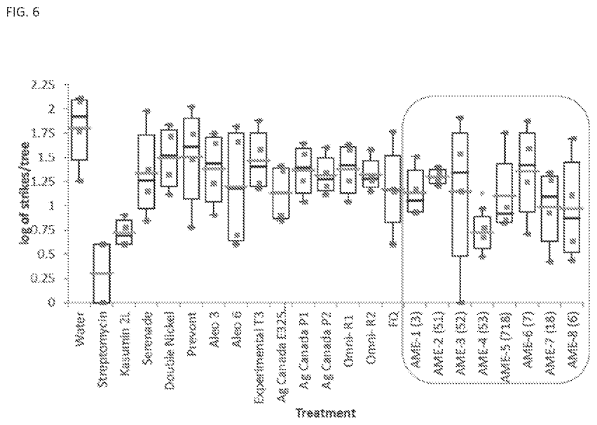

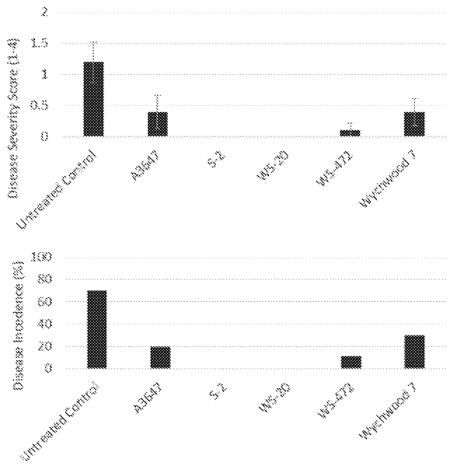

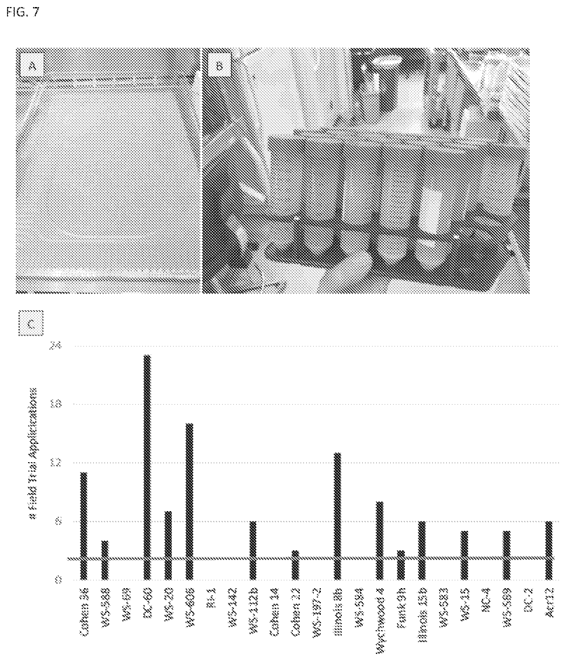

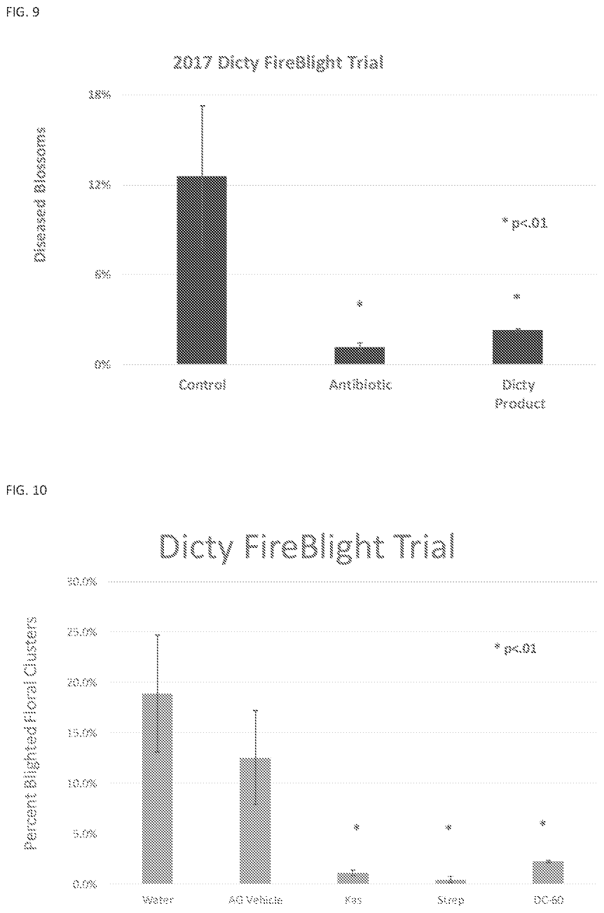

Two pairs/combinations that showed 3-log reduction in Ea titer in planta were identified. Apple seedlings were grown in a growth chamber under field relevant temp, humidity, and light conditions (FIG. 6). One pairwise combination of Dicty with DC-60 and Illinois 15b was tested in a field trial (FIGS. 9-10). This combination provided moderate disease control that was comparable to that of Illinois 15b alone. The data indicate that this combination was not antagonistic in the field.

### Develop Methods for Spore Production and Packaging

New production methods and different bacterial prey were used to improve yield. The highest-yielding method resulted from growing Dicty on live (nonpathogenic) E. coli (Br) on a custom agar formulation with optimized ionic strength on 13″×18″ plastic sheets. Using this method, more than 2.3×10¹⁰ spores were produced from 14 Dicty strains. This number of spores permitted testing of each of 14 strains under standard field trial parameters (3 applications to 4 trees at a concentration of 1×10⁶ spores per ml).

Three methods, based on published literature and consultation with experts, that are compatible with commercial application were tested: simple desiccation, vacuum drying, and lyophilization¹. Desiccation and lyophilization proved difficult to perform consistently without significant investment in equipment and method development. Vacuum drying can be performed quickly, returned consistent yields of viable spores and is amenable to commercial application methods used in the field.

None of the 10 probiotic bacteria examined supported growth and development of Dicty into spores. That is why nonpathogenic E. coli were used in scaling up spore production and preservation. Using the methods described herein, 3×10¹² (20 billion) spores were produced.

A simple, inexpensive, and convenient large-scale method to vacuum dry spores in nonfat dry milk while maintaining spore viability was developed using the protocol described in methods above. Dicty were able to be concentrated and then vacuum-dried in standard laboratory conical tubes. Additionally, the spores remain viable in water with small amounts of Silwet surfactant for at least 6 months.

Dicty strains were tested for their ability to feed on Xc pv. vesicatoria and Ps pv. tomato by conducting in vitro quantitative feeding assays on biofilm-enmeshed bacteria under agriculturally relevant conditions. Strains originating from California and Florida were identified, as these are the top two US states in terms of tomato production.

Of these strains, 36 fed robustly on a strain of Ps pv. Tomato. No strains were found to robustly consume Xc pv. Vesicatoria. However, 16 strains were found to show some Dicty development or feeding on Xc pv. vesicitoria.

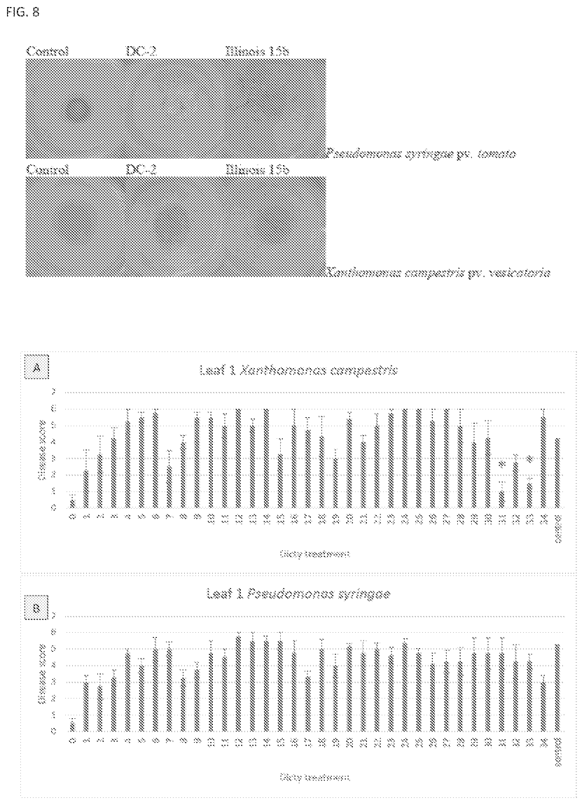

Strains were grown on polycarbonate membranes to encourage biofilm formation for 2 days, and then Dicty were applied to the Biofilm. It was determined that all of the 36 strains tested were able to consume biofilm-enmeshed Ps pv. tomato, but again saw no strains that were able to completely consume biofilm enmeshed Xc pv. vesicatoria, only limited feeding and peripheral development. (FIG. 8).

Top-performing Dicty strains from above were tested in planta for their ability to suppress induced bacterial disease on tomato seedlings inoculated with either Xc pv. vesicatoria or Ps pv. Tomato. A growth chamber with shelves, LED lighting, and constructed Ventilation was developed that was able to control temperature, humidity, and day length, accommodating 48 flats, or 864 plantlets at a time.

Eight cultivars were tested for their susceptibility to Xc pv. vesicatoria and Ps pv. tomato by spray-inoculating seedlings with 1×10⁸ cfu/mL of bacteria and observing disease phenotypes including height, leaf spot/speck, and stem canker, qualitatively assessing the severity of the leaf or stem injury. The tomato cv. Rutgers had the most consistent disease manifestation of the 8 tested cultivars, with scorable stem and leaf phenotypes and was used for subsequent assays (FIG. 9).

Using an in vitro viability assay, Dicty growth behavior with and without Regalia® biofungicide (Marrone Bio Innovations, Inc., Davis, Calif.) was assessed. It was found that the two top candidate strains from 2017 FB field trials, DC60 and Illinois 15b, showed no differences in growth upon the addition of Regalia®. Additional products, including MBI's Stargus® and an adjuvant, Biolink®, were tested and found to have no effect on Dicty's growth behavior in vitro.

Based on data from a field trial, it was determined that the most effective treatment had 60× the Dicty of a standard treatment.

TABLE 1

| | | Bacterial | |
|---|---|---|---|
| | | recovery | |
| | Disease severity | log10 | Dicty |
| | % reduction vs. control | reduction | recovery |
| Ea1189 control | 0.0 | 0.00 | NA |
| Illinois 15b | 100.0 | 0.24 | + |
| RI-1 | 76.2 | 0.39 | + |
| WS-197-2 | insect contamination | 0.24 | + |
| WS-588 | 82.1 | 0.60 | + |
| WS-606 | 82.1 | 0.28 | + |
| Cohen 22 | 52.4 | 0.43 | + |
| S-2 | 14.3 | 0.10 | + |
| WS-589 | 14.3 | 0.19 | + |
| WS-112b | 64.3 | 0.66 | + |
| WS-583 | −31.0 | 0.08 | + |
| NC-4 | 76.2 | 0.20 | + |
| WS-69 | −14.3 | 0.38 | + |
| WS-28-1 | −90.5 | 0.79 | + |
| WS-331a | 64.3 | 0.30 | + |
| WS 521 | −28.6 | 0.00 | + |
| Wychwood 4 | 71.4 | 0.44 | ? |
| WS-380b | 0.0 | 0.21 | + |
| WS-567 | −14.3 | −0.01 | + |
| WS-15 | 88.1 | 0.16 | + |
| WS-20 | −57.1 | 0.80 | + |
| WS-28 | −142.9 | 1.28 | + |
| WS-142 | −185.7 | 0.83 | + |
| WS-647 | 0.0 | 0.63 | + |
| Funk 9h | 14.3 | 0.24 | + |
| Acr12 | −128.6 | −0.16 | + |
| DC-2 | −66.7 | −0.05 | + |
| DC-60 | −14.3 | 0.63 | + |
| WS-584 | −7.1 | 0.32 | + |
| Cohen 14 | −14.3 | 0.33 | + |
| Cohen 36 | 46.4 | 0.72 | + |
| Illinois 8b | −142.9 | 0.04 | + |

### References

1 Raper, K. B. and A. W. Rahn (1984). The Dictyostelids. Princeton, N.J., Princeton University Press.
2 Bonn, W. G. and T. van der Zwet (2000). Distribution and economic importance of fire blight. Fire blight: the disease and its causative agent, Erwinia amylovora. J. L. Vanneste. Wallingford, Oxon, UK; New York, N.Y., CABI Pub.: 37-53.
3 Longstroth, M. (2001). “The 2000 fire blight epidemic in southwest Michigan apple orchards.” The Compact Fruit Tree 34: 16-19.
4 Vanneste, J. L. (2000). Fire blight: the disease and its causative agent, Erwinia amylovora. Wallingford, Oxon, UK; New York, N.Y., CABI Pub.
5 Mansfield et al. (2012). “Top 10 plant pathogenic bacteria in molecular plant pathology.” Molecular Plant Pathology 13(6): 614-629.
6 Merritt, J. H., et al. (2005). “Growing and analyzing static biofilms.” Current Protocols in Microbiology Chapter 1: Unit 1B 1.
7 Koczan, 2011 #2028 Koczan, J. M., et al. (2011). “Cell surface attachment structures contribute to biofilm formation and xylem colonization by Erwinia amylovora.” Applied and Environmental Microbiology 77(19): 7031-7039.
8 Koczan, J. M., et al. (2009). “Contribution of Erwinia amylovora exopolysaccharides amylovoran and levan to biofilm formation: implications in pathogenicity.” Phytopathology 99(11): 1237-1244.
9 Zhao, Y., et al. (2009). “Construction and analysis of pathogenicity island deletion mutants of Erwinia amylovora,” Can J Microbiol 55(4): 457-464.
10 McManus, P. S., et al. (2002). “Antibiotic use in plant agriculture.” Annu Rev Phytopathol 40: 443-465.
11 Zhao, Y., et al. (2005). “Identification of Erwinia amylovora genes induced during infection of immature pear tissue.” Journal of bacteriology 187(23): 8088-8103.
12 Sundin, G. W., et al. (2009). “Field evaluation of biological control of fire blight in the eastern United States.” Plant Disease 93(4): 386-394.
13 USDA ERS Tomatoes. www.ers.usda.gov/topics/crops/vegetables-pulses/tomatoes.aspx
14 Santos, et al., (2018) “Xanthomonas citri T6SS mediates resistance to Dictyostelium predation and is regulated by an ECF σ factor and cognate Ser/Thr kinase” Environ Microbiol. 20(4):1562-1575.

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure which are obvious to those skilled in the relevant fields are intended to be within the scope of the following claims.

We claim:

1. A method of treating a bacterial infection in a plant, wherein said bacteria is selected from the group consisting of Erwinia amylovora (Ea), Clavibacter michiganensis subsp. Michiganensis (Cmm), Xanthomonas campestris, and Pseudomonas syringae, comprising: contacting said plant with a composition comprising one or more species of amoebae.

2. The method of claim 1, therein said bacteria is present as a biofilm.

3. The method of claim 1, wherein said composition comprises two or more species of amoebae.

4. The method of claim 1, wherein said amoebae are a Dictyostelium sp.

5. The method of claim 4, wherein said Dictyostelium sp is selected from the group consisting of Illinois 15b, RI-1, WS-606, Cohen 22, NC-4, WS-28-1, WS-331a, Wychwood 4, WS-15, WS-20, WS-28, WS-142, WS-647, DC-60, and Cohen 36.

6. The method of claim 5, wherein said amoebae are WS606+Illinois 15b or DC60+Illinois 15b.

7. The method of claim 1, wherein said composition further comprises a non-amoebae anti-microbial agent.

8. The method of claim 7, wherein said anti-microbial agent is selected from the group consisting of a pesticide, an insecticide, a fertilizer, an herbicide, and a fungicide.

9. The method of claim 1, wherein said treating reduces one or more signs or symptoms of infection selected from the group consisting of blossom blight, cankers, and necrosis.

10. A composition, kit, or system, comprising:
a) one or more species of amoebae, wherein said amoebae are a Dictyostelium sp. selected from the group consisting of Illinois 15b, RI-1, WS-606, cohen 22, NC-4, WS-28-1, WS-331a, Wychwood 4, WS-15, WS-20, WS-28, WS-142, WS-647, DC-60, and Cohen 36; and
b) a carrier.

11. The composition, kit, or system of claim 10, wherein said composition further comprises an anti-microbial agent selected from the group consisting of pesticide, an insecticide, a fertilizer, an herbicide, and a fungicide.

12. The composition, kit, or system of claim 10, wherein said amoebae are WS606+Illinois 15b or DC60+Illinois 15b.

13. The composition, kit, or system of claim 10, wherein said ameba are present as spores.

14. The composition, kit, or system of claim 10, wherein said ameba are lyophilized.

15. The composition, kit, or system of claim 10, wherein said composition is formulated as a dip, seed dressing, stem injection, spray, or mist.

16. The composition, kit, or system of claim 10, wherein amoebae are present in unit dosage form.

17. The composition, kit, or system of claim 13, wherein said amoebae are at a concentration of 1×10⁶ spores per ml.
